(19) United States
(12) Patent Application Publication
(10) Pub. No.: US 2014/0255328 A1
(43) Pub. Date: Sep. 11, 2014

(54) Hydrothermal Synthesis of Zinc Phlogopite

(71) Applicant: BASF SE, Ludwigshafen (DE)

(72) Inventors: Meaghan Clark McGuire, Nanuet, NY (US); Ivor Bull, Hopewell Junction, NY (US); Geoffrey Mark Johnson, Wappingers Falls, NY (US)

(21) Appl. No.: 14/204,151

(22) Filed: Mar. 11, 2014

Related U.S. Application Data
(60) Provisional application No. 61/776,228, filed on Mar. 11, 2013.

(51) Int. Cl. C08K 3/34; A61K 8/27; C09D 11/00; C09D 11/16; C08K 3/22; C09D 7/12

(52) U.S. Cl. CPC C08K 3/34; C08K 3/22; C09D 7/1216; C09D 11/322; C09D 11/17; A61K 8/27
USPC 424/63; 106/417; 524/443; 423/328.3; 428/402

(57) ABSTRACT

This invention relates to a synthetically derived zinc phlogopite platelets, of superior platelet diameter, effect pigments comprising such synthetically derived platelets and methods of forming said substrates. More specifically the disclosure describes an improved hydrothermal synthesis of zinc phlogopite suitable as a platelets for interference pigments, barrier and flame retardant applications.

This application claims the benefit of U.S. provisional application Ser. No. 61/776,228 filed Mar. 11, 2013 incorporated entirely by reference.

### TECHNICAL FIELD

This application is directed to a synthetic zinc phlogopite platelet and improved methods of making synthetic zinc phlogophite platelets via low temperature, low pressure hydrothermal conditions.

### BACKGROUND ART

Natural mica is mined in the presence of sand, kaolin, feldspar and other silicates and will contain various impurities such as iron oxides and heavy metals. Because of the presence of these additional impurities, mica is often discolored. This discoloration is of course, an undesired characteristic of the natural material particularly when the mica is used as a platelet, core or substrate for interference pigments, barrier coatings and the like.

Furthermore natural mica must be ground to produce flakes. This grinding does not allow for tight control of the smoothness of the mica surface, stepped characteristics and the thinness of the flake. Accordingly, the flakes often have imperfect edges, faces and less specular reflection (edge scattering).

As a result, synthetic alternatives have long been desired which would provide high purity formation and high aspect ratio. Synthetic mica containing fluoride is well known in the art and is most commonly prepared via a melt reaction method. Synthetic fluoride containing mica powder of high purity has been prepared by mixing compounds containing oxides and/or fluorides of potassium, sodium, magnesium, aluminum and silicon at a predetermined ratio, melting, crystallizing, cooling, and then mechanically pulverizing. However, even synthetic mica prepared via solid phase synthesis is problematic. The solid phase synthesis requires grinding and the grinding process leads to stepping, lack of flake thickness control and variability of plate diameter size. The natural or synthetic mica normally consists of platelets having a thickness of about 500 to 600 nm and a defined particle size distribution.

Ideally, a synthetic pathway is needed which would lead directly to mica characterized by low thickness parameters and less specular reflection (edge scattering).

It is known to prepare synthetic phlogopite hydrothermally at high pressures and temperatures. For example, Yoder H. S. et al., Geochimica et Cosmochimica Acta, 1954, Vol. 6, pp. 157-185 teaches the formation of synthetic phlogopite at 1080° C. and 75,000 psi water vapour pressure. Frondel, C et al., The American Mineralogist, Vol. 51, 1966 teaches the formation of small particle size zinc containing micas via hydrothermal crystallization at high pressure (1000-3000 bars) and high temperatures (250° C. to 650° C.).

It is also known to prepare synthetic hydroxide containing mica via lower temperature hydrothermal methods. For example, Komarneni, S. et al., Clays and Clay Minerals, Vol. 51. p. 693 and Perrotta, A. et al., J. American Mineralagist, Vol. 60, p. 152 describe hydrothermal methods of forming a zinc containing mica of the hydroxide phlogopite type. Korean Patent Publication No. 20070111271 teaches hydrothermal preparation of magnesium phlogopite.

However, the processes presented therein are unsatisfactory in regard to the morphology of the platy material, smoothness of the platelet, transparency, the purity of crystal formation, the length of preparation times, diameter, thinness of the platy substrate and temperatures and pressures required.

Accordingly, there is a pressing need in the art to devise a method of preparation of synthetic mica, especially zinc phlogopite production wherein single crystals are produced (no grinding necessary) and major dimensions of the mica platlets, such as thickness and/or platelet diameter can be adequately controlled and to carry out this method using low temperatures and pressures.

Furthermore, U.S. provisional application No. 61/776,228 filed on Mar. 11, 2013 and herein incorporated entirely by reference teaches the hydrothermal preparation of synthetic zinc phlogopite of high aspect ratio using habit modifiers. The present application also teaches hydrothermal preparation of synthetic zinc phlogopite but the present application preparation does not require the presence of a habit modifier.

The applicants have discovered that by changing certain process variables of the hydrothermal process, it is possible to make synthetic zinc phlogopite platelets characterized by a diameter which significantly exceeds known hydrothermal methods of preparing zinc phlogopite.

### SUMMARY OF THE INVENTION

The present applicants have discovered a method for producing a synthetic zinc phlogophite hydrothermally at low temperatures and pressures which meet the above needs and exceed diameters of previously formed synthetic zinc phlogopite platelets via hydrothermal methods.

Thus this application claims a synthetic zinc phlogopite platelet of formula (1)

IZn₃(AlSi₃O₁₀)(X)₂ (1)
wherein
I is an interlayer monovalent cation selected from the group consisting of K⁺, Na⁺ and Li⁺, NH₄⁺, preferably K⁺;
and
X is independently fluoride, hydroxide or fluoride and hydroxide, preferably hydroxide, wherein the platelet is characterized by a diameter of greater than 2 microns, preferably at least 3 microns and most preferably at least 4 microns.
   Preparation of the synthetic zinc phlogopite platelet of formula (1),

IZn₃(AlSi₃O₁₀)(X)₂ (1)
wherein
I is an interlayer monovalent cation selected from the group consisting of K⁺, Na⁺ and Li⁺, NH₄⁺, preferably K⁺;
and
X is independently fluoride, hydroxide or a combination of both hydroxide and fluoride, preferably hydroxide;
is formed comprising the steps of:
forming a reaction mixture comprising
an I source selected from the group consisting of sodium, potassium, ammonium and lithium, preferably potassium;
   an aluminum source;
   a silicon source;
   a zinc source;
   optionally a fluoride source and/or hydroxide source, preferably a hydroxide source;
   and
   optionally seed crystals of a preformed phlogopite crystals,
hydrothermally treating said reaction mixture under basic conditions at a temperature ranging from about 125 to about 250° C., preferably 150° C. to about 225° C.
and
a pressure ranging from about 50 to about 400 psi, preferably about 100 psi to about 220 psi;
to form the zinc phlogopite platelet of formula (1);
and
optionally isolating the formed platelet,
wherein the silicon source is colloidal silica.
Additionally, the above synthetic zinc phlogopite platelet is envisioned as a substrate for an effect pigment.
The effect pigment comprises the above synthetic zinc phlogopite platelet comprising
(a) a layer of a dielectric material, especially a metal oxide, having a high index of refraction; and/or
(b) a metal layer, especially a thin semi-transparent metal layer.

### SHORT DESCRIPTION OF THE DRAWINGS

FIG. 1—shows the morphology of the zinc phlogophite formed in example 1;

FIG. 2—shows the thickness of the particles formed in example 2;

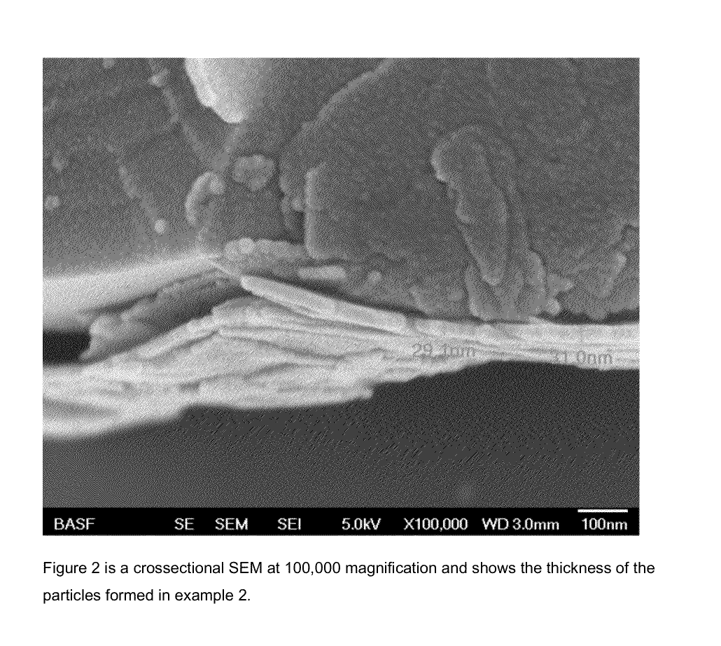

FIG. 3—shows the platelet formed in example 2.

### DETAILED DESCRIPTION OF THE INVENTION

### Definitions

The term “hydrothermal process” as used herein means a process that allows platelets of material such as zinc phlogopite to grow in a solvent at temperatures and pressures which allow for the at least partial dissolution or dispersion of precursor materials.

The terms “synthetically derived” means the zinc phlogopite is formed synthetically, i.e. by a controlled chemical reaction, specifically a hydrothermal reaction. The hydrothermal reaction conditions disclosed herein are those characterized by low temperature and low pressures.

The terms “low temperature” and “low pressure” when used to describe the hydrothermal process conditions means for purposes of this application temperatures ranging from 125 to about 250° C., preferably 150° C. to about 225° C. and pressures ranging from about 50 to about 400 psi, and preferably about 100 psi to about 220 psi.

The term “platelet, platy, plate-like and flakey” are typical terms used in the art and is understood to mean that the platy substrates have a diameter which is greater than the thickness of the substrate, such as platelets (flakes).

The term “aspect ratio” refers to the ratio of the maximum dimension (diameter or d₅₀) to the minimum dimension (thickness) of a particle. In other words when the term “aspect ratio” is used herein the ratio of diameter/particle thickness is meant.

The diameter is preferably defined as the d₅₀ particle size distribution determined via static light scattering using a Malvern Mastersizer® Hydro2000S. The thickness of the platelet is determined via Scanning Electron Microsope (SEM).

The reaction mixture will typically be an aqueous dispersion, solution, slurry or gel of the starting materials.

The reaction may be run in a sealed or unsealed vessel.

Phlogopite

Phyllosilicates are silicate minerals having the tetrahedral silicate groups linked in sheets, each group containing four oxygen atoms, three of which are shared with other groups so that the ratio of silicon atoms to oxygen atoms is two to five. Mica is a subset of phyllosilicates. Phlogopite is a subset of mica and zinc phlogopite is a subset of phlogopites.

Phlogopite is a mica which has a layered structure most commonly comprising magnesium aluminum silicate sheets weakly bonded together by layers of alkali ion (sodium, lithium or potassium ions). For example, potassium containing phlogopite (KMg₃AlSi₃O₁₀(F,OH)₂ has potassium ions weakly bonding the magnesium aluminum silicate sheets.

The most preferred mica of formula (1) is zinc containing phlogopite. For example, KZn₃AlSi₃O₁₀(OH)₂, KZn₃AlSi₃O₁₀(OH,F) and KZn₃AlSi₃O₁₀(F)₂ may be made by the hydrothermal process herein disclosed.

### The Synthetic Zinc Phlogophite Platelet

The diameter of a platelet may be defined preferably as the D₅₀ particle size distribution determined via static light scattering using a Malvern Mastersizer® Hydro2000S. The thickness of the platelet is determined via cross sectional Scanning Electron Microsope (SEM).

The synthetic zinc phlogopite platelet is substantially transparent, that is it transmits at least 92% light, preferably 95% light and most preferably 98% light.

### Identification of the Crystal Form of the Phyllosillicate Crystals

Identification of the zinc phlogopite crystals are carried out via X-ray diffraction. The Powder X-ray scan (PXRD) is performed using CuK_{α} radiation source.

It is presently preferred that the diameter of the hydrothermally prepared zinc phlogopite, range from at least 2.5 microns to about 1 mm with a more preferred range of about 3 microns to about 60 microns, especially about 3 microns to about 30, 40 or 50 microns or about 3 microns to about 30 or 40 microns.

The thickness of the synthetic zinc phlogopite will for example range from about 10 nm to about 500 nm, preferably about 20 nm to about 400 nm. For example the thickness may range from most preferably about 10 nm to about 150 nm, especially about 15 nm to about 100 nm or about 15 nm to about 80 nm.

### Morphology of Zinc Phlogopite Formed According to the Invention

The morphology of the synthetic zinc phlogopite is platelet like.

### Particle Size Distribution

A particularly useful means of characterizing the size distribution of a mass of synthetic platelets produced is by specifying the platelet size of the lowest 10 vol. %, 50 vol. %, and 90 vol. % of platelets along the Gaussian curve. This classification can be characterized as the d₁₀, d₅₀, and d₉₀ values of the platelet size distribution. Thus, a platelet having a d₁₀ of a certain size means that 10 vol. % of the platelet particles has a size up to that value. Thus, the size distribution of the mica-based platelets can be described as follows: 10 volume % of the platelets have a size of up to and including 10 microns, 50 volume % of the platelets have a size up to and including 22 microns, and 90 volume % of the platelets have a size up to and including 45 microns for example.

For example the synthetically derived zinc phlogopite platelets are preferably characterized by a d₅₀ ranging from about 2.5, 3 or 4 microns to about 60 microns, most preferably about 5 or 6 microns and above, especially the synthetically derived zinc phlogopite platelets may be characterized by a d₅₀ of about 2.5-5 microns to about 30 microns or 40 microns.

The platelet may of course be classified by means of various methods, such as gravity sedimentation, sedimentation in a decanter, sieving, use of a cyclone or hydrocylone, spiral classifying or a combination of two or more these methods. A method such as sieving, for example, may also be used in a plurality of successive steps. Classification may shift the distribution of platelet toward larger or smaller diameters.

### Hydrothermal Process Variables

As explained above the term “hydrothermal process” as used herein means a process that allows crystals of the zinc phlogopite platelet to grow in a solvent at low temperature and low pressure.

Preferably the preparation of the synthetic zinc phlogopite platelet of formula (1)

IZn₃(AlSi₃O₁₀)(X)₂ (1)
comprises the steps of:
forming a reaction mixture comprising
   I source selected from the group consisting of sodium, potassium, ammonium and lithium, preferably potassium;
   an aluminum source;
   a silicon source;
   a zinc source;
   optionally a fluoride source and/or hydroxide source, preferably hydroxide;
   optionally a habit modifier and/or seed crystals of a preformed phlogopite;
hydrothermally treating said reaction mixture at a temperature ranging from about 150 to about 250° C. and a pressure ranging from about 50 to about 400 psi under basic conditions to form the platelets of the synthetic zinc phlogopite;
and
optionally isolating the formed synthetic phlogopite
wherein the silicon source is colloidal silica and the silicon source, zinc source and aluminum sources are present in the reaction mixture at molar ratios consistent with the formed zinc phlogopite.

The base may for example be derived from common inorganic bases such as potassium hydroxide, sodium hydroxide, lithium hydroxide, sodium carbonate, lithium carbonate, ammonium hydroxide, and potassium carbonate and organic bases such as tripropylammonium hydroxide, tetramethyl ammonium hydroxide, triethanolamine and diethanolamine.

Preferably the base is an inorganic base and is selected from the group consisting of potassium hydroxide, lithium hydroxide, lithium carbonate, sodium hydroxide, ammonium hydroxide, sodium carbonate and potassium carbonate.

The initial reaction mixture should be basic. Typically the pH of the initial reaction mixture will range from about 9 to about 14, preferably the pH will range from about 12 to about 14, and most preferably will range for about 12.5 to about 14.

As explained above I is an interlayer monovalent cation selected from the group consisting of K⁺, Na⁺, NH₄⁺ and Li⁺, preferably K⁺ and Na⁺. The source for this cation may be from the base used to ensure basic reaction conditions of the hydrothermal process. For example, bases which would provide a K⁺, Na⁺, NH₄⁺ or Li⁺ may be potassium hydroxide, sodium hydroxide, Na₂O lithium hydroxide, sodium carbonate, ammonium hydroxide, lithium carbonate, Li₂O, potassium carbonate and K₂O

Preferably the interlayer monovalent cation is K⁺,

The aluminum source may be selected from elemental aluminum Al⁰, salts of aluminum (organic or inorganic), oxide and hydrates thereof. For example, the aluminum source may be selected from the group consisting of Al(NO₃)₃(aluminum nitrate), K₂Al₂O₄(potassium aluminate), Na₂Al₂O₄ (sodium aluminate) Al(OH)₃(aluminum hydroxide), Al₂O₃, psuedoboehmite, aluminum isopropoxide, Al(C₂H₃O₂)₃, AlBr₃, AlCl₃, Al(C₆H₅O₇) (aluminum citrate), AlF₃, Al(CHO₂)₃ (aluminum formate), Al₂(SO₄)₃, AlOOH (aluminum hydroxide oxide) and hydrates thereof.

Preferably the aluminum source is selected from Al(NO₃)₃(aluminum nitrate), K₂Al₂O₄ (potassium aluminate), Na₂Al₂O₄, Al₂(SO₄)₃, Al(OH)₃, AlOOH and hydrates thereof and most preferably the aluminum source is selected from group consisting of Al(NO₃)₃(aluminum nitrate), Al₂(SO₄)₃, K₂Al₂O₄ (potassium aluminate) and hydrates thereof.

The most preferable aluminum source is potassium aluminate (K₂Al₂O₄) and Al(NO₃)₂.

The silica sources are typically derived from hydrates of SiO₂, colloidal SiO₂, sodium metasilicate, sodium silicate, potassium metasilicate, potassium silicate, lithium metasilicate, lithium silicate, kaolin, fumed silica, talc, H₂SiO₃ and tetraethyl orthosilicate.

Colloidal SiO₂ is most preferred. It has been discovered that when colloidal silica is used as the silicon source, the morphology of the particles are quite different than those of known hydrothermal methods for zinc phlogopite synthesis. Additionally, using colloidal silica appears to increase the diameter size of the platelets significantly.

Most preferably the synthetic zinc phlogopite platelet of formula (1),

IZn₃(AlSi₃O₁₀)(X)₂ (1)
wherein
I is an interlayer monovalent cation selected from the group consisting of K⁺, N⁺, Li⁺ and NH₄⁺, preferably K⁺;
and
X is independently fluoride or hydroxide, a combination of fluoride and hydroxide, preferably hydroxide;
is prepared comprising the steps of:
forming a reaction mixture comprising
   an I source selected from the group consisting of sodium, potassium and lithium, preferably potassium;
   an aluminum source, preferably Al(NO₃)₃(aluminum nitrate), K₂Al₂O₄(potassium aluminate), Na₂Al₂O₄, Al(OH)₃, AlOOH, Al₂(SO4)₃ and hydrates thereof and most preferably aluminum source is potassium aluminate (K₂Al₂O₄) and Al(NO₃)₃;
   a silicon source;
   a zinc source;
   optionally a fluoride source and/or hydroxide source, preferably hydroxide;
   and
   optionally a seed crystals of a preformed phlogopite crystals,
hydrothermally treating said reaction mixture under basic conditions at a temperature ranging from about 125 to about 250° C., preferably 150° C. to about 225° C.
and
a pressure ranging from about 50 to about 400 psi, preferably about 100 psi to about 220 psi;
to form the zinc phlogopite platelet of formula (1);
and
optionally isolating the formed platelet
and the silicon source is colloidal silica and the silicon source, zinc source and aluminum sources are present in the reaction mixture at molar ratios consistent with the formed zinc phlogopite.

### Zinc Source

The zinc source may be elemental, any salt (organic or inorganic), hydrate or oxide thereof. The zinc source may be selected from the group consisting of Zn⁰, ZnSO₄, Zn(NO₃)₂, ZnCl₂, Zn(C₂H₃O₂)₂ (zinc acetate), ZnCO₃, Zn(CHO₂)₂ (zinc formate), ZnBr₂, zinc oxide, ZnI₂ and hydrates thereof. ZnSO₄ is the preferable source for zinc.

Especially the process for preparing the synthetic zinc phlogopite platelet of formula (1),

IZn₃(AlSi₃O₁₀)(X)₂ (1)
wherein
I is an interlayer monovalent cation selected from the group consisting of K⁺, Na⁺ and Li⁺ and NH₄⁺, preferably K⁺;
and
X is independently fluoride or hydroxide, a combination of fluoride and hydroxide, preferably hydroxide;
is prepared comprising the steps of:
forming a reaction mixture comprising
   an I source selected from the group consisting of sodium, potassium, ammonium and lithium, preferably potassium;
   an aluminum source, selected from the group consisting of Al(NO₃)₃ (aluminum nitrate), K₂Al₂O₄ (potassium aluminate), Na₂Al₂O₄, Al(OH)₃, AlOOH, Al₂(SO4)₃ and hydrates thereof and preferably aluminum source is potassium aluminate (K₂Al₂O₄) and Al(NO₃)₃;
   a silicon source;
   a zinc source selected from the group consisting of Zn⁰, ZnSO₄, Zn(NO₃)₂, ZnCl₂, Zn(C₂H₃O₂)₂ (zinc acetate), ZnCO₃, Zn(CHO₂)₂ (zinc formate), ZnBr₂, zinc oxide, ZnI₂ and hydrates thereof, preferably ZnSO₄;
   optionally a fluoride source and/or hydroxide source, preferably an hydroxide source;
   and
   optionally seed crystals of a preformed phlogopite crystals,
hydrothermally treating said reaction mixture under basic conditions at a temperature ranging from about 125 to about 250° C., preferably 150° C. to about 225° C.
and
a pressure ranging from about 50 to about 400 psi, preferably about 100 psi to about 220 psi;
to form the zinc phlogopite platelet of formula (1);
and
optionally isolating the formed platelet
and the silicon source is colloidal silica and the silicon source, zinc source and aluminum sources are present in the reaction mixture at molar ratios consistent with the formed zinc phlogopite.

### Fluoride Source

The optional fluoride source is for example HF, NH₄F, NaF, K₂SiF₆, KF and MgF₂.

It is preferable not to include a fluoride source.

The water content during the hydrothermal reaction may vary from about 60 to about 98 wt. percent. Thus the wt. % reagents will normally range from about 2 wt. % to about 40 wt. percent, preferably from about 4 wt. % to about 35 wt. % and most preferably about 5 wt. % to about 30 wt. %. The weight % is based on the total weight of the reaction mixture.

### Hydroxide Source

The hydroxide source may come from the bases such as potassium hydroxide, sodium hydroxide, lithium hydroxide, ammonium hydroxide, and organic bases such as tripropylammonium hydroxide and tetramethyl ammonium hydroxide.

It is preferable to have a hydroxide source. The base may provide the hydroxide source.
Thus the process for making the zinc phlogopite of formula (1) comprises the steps of:
forming a reaction mixture comprising
   an I source selected from the group consisting of Na⁺, K⁺, NH₄⁺ and Li⁺, preferably K⁺;
   an aluminum source;
   a silicon source;
   a zinc source;
   optionally a fluoride source and/or an hydroxide source; preferably an hydroxide source;
   and
   optionally seed crystals of a preformed phlogopite crystals,
hydrothermally treating said reaction mixture under basic conditions at a temperature ranging from about 125 to about 250° C., preferably 150° C. to about 225° C.
and
a pressure ranging from about 50 to about 400 psi, preferably about 100 psi to about 220 psi;
to form the zinc phlogopite platelet of formula (1);
and
optionally isolating the formed platelet
and the silicon source is colloidal silica.

### Seeding

Seeding of the hydrothermal reaction may be desirable with a previously formed mica. The amount of seeding making up the reaction mixture may range from about 1 to 6 wt. percent of the calculated mica platelet, preferably the calculated phlogopite product. For example if the product intended is a phlogopite, the hydrothermal reaction may be seeded with a wt. % of phlogopite seed crystal ranging from about 0.1 to about 10 wt. %, preferably 0.5 to about 8 wt. %, most preferably 1 to about 6 wt. % of the theoretical product formed.

### Time, Temperature and Pressure

The hydrothermal reaction mixture is typically heated to the appropriate temperature, about 150 to about 250° C., then held at the appropriate temperature from about 2 to about 100 hours, more typically about 10 to 90 hours or most typically about 20 to about 85 hours.

The pressure conditions for carrying out the hydrothermal reactions will vary depending upon the platelet but will typically vary from about 50 psi to about 400 psi, more typically about 75 psi to about 300 psi, most typically from about 85 to about 250 psi.

The hydrothermal process for production of the zinc phlogophite may be done under static or stirring/mixing conditions.

### Stoichiometric Conditions or Non-Stoichiometric Conditions for Hydrothermal Preparation

The hydrothermal preparation of the zinc phlogopite can be done under stoichiometric conditions or non-stoichiometric conditions. It is preferable that the reaction is run under stoichiometric conditions.

Stoichiometric conditions means for purposes of this application, that the starting materials, in particular zinc source, silicon source, and aluminum source are present at the start of the reaction at the same molar ratios of the final product, the zinc phlogopite.

The applicants have discovered that stoichiometric conditions make a very significant difference in the size, shape and size distribution of the hydrothermally formed zinc phlogopite platelets.

Thus the synthetic zinc phlogopite platelet of formula (1) is preferably prepared

IZn₃(AlSi₃O₁₀)(X)₂ (1)
wherein
I is an interlayer monovalent cation selected from the group consisting of K⁺, Na⁺, NH₄⁺ and Li⁺, preferably K⁺; and
X is independently fluoride or hydroxide, a combination of fluoride and hydroxide, preferably OH;
comprising the steps of:
forming a reaction mixture comprising
   an I source selected from the group consisting of sodium, potassium, ammonium, and lithium, preferably potassium;
   an aluminum source, preferably selected from the group consisting of Al(NO₃)₃(aluminum nitrate), K₂Al₂O₄(potassium aluminate), Na₂Al₂O₄, Al(OH)₃, AlOOH, Al₂(SO₄)₃ and hydrates thereof and most preferably selected from the group consisting of potassium aluminate (K₂Al₂O₄) and Al(NO₃)₃;
   a silicon source,
   a zinc source, preferably selected from the group consisting of Zn⁰, ZnSO₄, Zn(NO₃)₂, ZnCl₂, Zn(C₂H₃O₂)₂ (zinc acetate), ZnCO₃, Zn(CHO₂)₂ (zinc formate), ZnBr₂, zinc oxide, ZnI₂ and hydrates thereof most preferably ZnSO₄
   optionally a fluoride source and/or an hydroxide source, preferably an hydroxide source;
   and
   optionally seed crystals of a preformed phlogopite crystals,
hydrothermally treating said aqueous gel, dispersion or solution under basic conditions at a temperature ranging from about 125 to about 250° C., preferably 150° C. to about 225° C.
and
a pressure ranging from about 50 to about 400 psi, preferably about 100 psi to about 220 psi;
to form the zinc phlogopite platelet of formula (1);
and
optionally isolating the formed platelet and the silicon sources is colloidal silica and the silicon source, zinc source and aluminum source are present in the reaction mixture at molar ratios consistent with the formed zinc phlogopite.

### Applications for Hydrothermally Produced Mica

There are many applications for the presently disclosed zinc phlogopite. For examples, zinc phlogopite is an excellent insulator, reinforcement material, solid lubricant, cosmetic extender, substrate or core for effect and interference pigments, barrier to gases (ie water vapor, CO₂ and oxygen) in packaging and paper applications and filler in resins providing heat resistance.

### Effect Pigment Using the Zinc Phlogopite

Effect pigments and their use in paints, ink-jet printing, for dyeing textiles, for pigmenting coatings, printing inks, plastics, cosmetics, glazes for ceramics and glass is well known in the art.

Such pigments having a core consisting of a transparent carrier material, such as, for example, natural, or synthetic mica, SiO₂, or glass, are known. Reference is made, for example, to Gerhard Pfaff and Peter Reynders, Chem. Rev. 99 (1999) 1963-1981.

The presently formed substrate, the synthetically derived phlogopite, is an especially suitable substrate, core or platelet for formation of an effect pigment.

One of the objects of the present invention is to develop pearlescent pigments on the basis of the presently hydrothermally produced zinc phlogopite with the disclosed diameters of 2.5 or about 3 microns or greater. The presently coated synthetic zinc phlogopite would exhibit the advantages of mica pigments (e.g. good application properties in a variety of binder systems, environmental compatibility and simple handling) with the possibility of realizing superior optical effects, i.e. to provide interference pigments, having high color strength and/or color purity.

This objective has been solved by pigments, comprising a plate-like substrate of the hydrothermally produced platelets of zinc phlogopite of platelet diameters of 2.5 microns or greater,

(a) a dielectric material, especially a metal oxide, having a high index of refraction; and/or
(a) a metal layer, especially a thin semi-transparent metal layer.

The pigment particles (coated core of synthetically produced phlogopite) generally have a diameter of from 2.5 microns to 1 mm, and an average thickness of <1 micron, such as for example thickness of 10 to about 150 nm, preferably about 15 to about 100 nm or most preferably about 15 to about 80 nm which contain a core of synthetically derived zinc phlogopite, having two substantially parallel faces, the distance between which is the shortest axis of the core. The core is either coated with a dielectric material, especially a metal oxide, having a high index of refraction, or a metal layer, especially a thin semi-transparent metal layer. Said layers can be coated with additional layers.

Suitable metals for the (semi-transparent) metal layer are, for example, Cr, Ti, Mo, W, Al, Cu, Ag, Au, or Ni. The semi-transparent metal layer has typically a thickness of between 5 and 25 nm, especially between 5 and 15 nm.

According to the present invention the term “aluminum” comprises aluminum and alloys of aluminum. Alloys of aluminum are, for example described in G. Wassermann in Ullmanns Enzyklopadie der Industriellen Chemie, 4. Auflage, Verlag Chemie, Weinheim, Band 7, S. 281 to 292. Especially suitable are the corrosion stable aluminum alloys described on page 10 to 12 of WO00/12634, which comprise besides of aluminum silicon, magnesium, manganese, copper, zinc, nickel, vanadium, lead, antimony, tin, cadmium, bismuth, titanium, chromium and/or iron in amounts of less than 20% by weight, preferably less than 10% by weight.

The metal layer can be obtained by wet chemical coating or by chemical vapor deposition, for example, gas phase deposition of metal carbonyls. The substrate is suspended in an aqueous and/or organic solvent containing medium in the presence of a metal compound and is deposited onto the substrate by addition of a reducing agent. The metal compound is, for example, silver nitrate or nickel acetyl acetonate (WO03/37993).

According to U.S. Pat. No. 3,536,520 nickel chloride can be used as metal compound and hypophosphite can be used as reducing agent. According to EP-A-353544 the following compounds can be used as reducing agents for the wet chemical coating: aldehydes (formaldehyde, acetaldehyde, benzalaldehyde), ketones (acetone), carbonic acids and salts thereof (tartaric acid, ascorbinic acid), reductones (isoascorbinic acid, triosereductone, reductine acid), and reducing sugars (glucose). However, it is also possible to use reducing alcohols (allyl alcohol), polyols and polyphenols, sulfites, hydrogensulfites, dithionites, hypophosphites, hydrazine, boron nitrogen compounds, metal hydrides and complex hydrides of aluminium and boron. The deposition of the metal layer can furthermore be carried out with the aid of a CVD method. Methods of this type are known. Fluidised-bed reactors are preferably employed for this purpose. EP-A-0741170 describes the deposition of aluminium layers by reduction of alkylaluminium compounds using hydrocarbons in a stream of inert gas. The metal layers can furthermore be deposited by gas-phase decomposition of the corresponding metal carbonyls in a heatable fluidised-bed reactor, as described in EP-A-045851. Further details on this method are given in WO93/12182. A further process for the deposition of thin metal layers, which can be used in the present case for the application of the metal layer to the substrate, is the known method for vapour deposition of metals in a high vacuum. It is described in detail in Vakuum-Beschichtung [Vacuum Coating], Volumes 1-5; Editors Frey, Kienel and Löbl, VDI-Verlag, 1995. In the sputtering process, a gas discharge (plasma) is ignited between the support and the coating material, which is in the form of plates (target). The coating material is bombarded with high-energy ions from the plasma, for example argon ions, and thus removed or atomised. The atoms or molecules of the atomised coating material are precipitated on the support and form the desired thin layer. The sputtering process is described in Vakuum-Beschichtung [Vacuum Coating], Volumes 1-5; Editors Frey, Kienel and Löbl, VDI-Verlag, 1995. For use in outdoor applications, in particular in the application in vehicle paints, the pigments can be provided with an additional weather-stabilising protective layer, the so-called post-coating, which simultaneously effects optimum adaptation to the binder system. Post-coatings of this type have been described, for example, in EP-A-0268918 and EP-A-0632109.

If pigments with metallic appearance are desired, the thickness of the metal layer is >25 nm to 100 nm, preferably 30 to 50 nm. If pigments with colored metal effects are desired, additional layers of colored or colorless metal oxides, metal nitrides, metal sulfides and/or metals can be deposited. These layers are transparent or semi-transparent. It is preferred that layers of high index of refraction and layers of low index of refraction alternate or that one layer is present, wherein within the layer the index of refraction is gradually changing. It is possible for the weathering resistance to be increased by means of an additional coating, which at the same time causes an optimal adaption to the binder system (EP-A-268918 and EP-A-632109).

In one preferred embodiment of the present invention, the interference pigments comprise materials having a “high” index of refraction, which is defined herein as an index of refraction of greater than about 1.65, and optionally materials having a “low” index of refraction, which is defined herein as an index of refraction of about 1.65 or less. Various (dielectric) materials that can be utilized including inorganic materials such as metal oxides, metal suboxides, metal fluorides, metal oxyhalides, metal sulfides, metal chalcogenides, metal nitrides, metal oxynitrides, metal carbides, combinations thereof, and the like, as well as organic dielectric materials. These materials are readily available and easily applied by physical, or chemical vapor deposition processes, or by wet chemical coating processes.

Optionally a SiO₂ layer can be arranged between the inventive phlogopite substrate and the materials having a “high” index of refraction. By applying a SiO₂ layer on the substrate the mica surface is protected against chemical alteration, such as, for example, swelling and leaching of mica components. The thickness of the SiO₂ layer is in the range of 5 to 200 nm, especially 40 to 150 nm. The SiO₂ layer is preferably prepared by using an organic silane compound, such as tetraethoxy silane (TEOS). The SiO₂ layer can be replaced by thin layers (thickness 1 to 20 nm) of Al₂O₃, Fe₂O₃ or ZrO₂.

Furthermore, the SiO₂-coated, or TiO₂-coated the synthetic zinc phlogopite flakes may, as described in EP-A-0 982 376, be coated with a nitrogen-doped carbon layer. The process described in EP-A-0 982 376 comprises the following steps:
(a) suspending the SiO₂, or TiO₂ coated synthetic mica flakes in a liquid,
(b) where appropriate adding a surface-modifier and/or a polymerization catalyst,
(c), before or after step (b), adding one or more polymers comprising nitrogen and carbon atoms, or one or more monomers capable of forming such polymers,
(d) forming a polymeric coating on the surface of the flakes,
(e) isolating the coated flakes and
(f) heating the coated flakes to a temperature of from 100 to 600° C. in a gaseous atmosphere.

The polymer may be a polypyrrole, a polyamide, a polyaniline, a polyurethane, a nitrile rubber or a melamine-formaldehyde resin, preferably a polyacrylonitrile, or the monomer is a pyrrole derivative, an acrylonitrile, a methacrylonitrile, a crotonitrile, an acrylamide, a methacrylamide or a crotonamide, preferably an acrylonitrile, methacrylonitrile or crotonitrile, most preferably an acrylonitrile.

Preferably, the flakes are heated in step (f) initially to from 100° C. to 300° C. in an oxygen-containing atmosphere and then to from 200 to 600° C. in an inert gas atmosphere.

The present invention therefore relates also to pigments based on the synthetic zinc phlogopite flakes according to the invention comprising over the entire surface of the silicon oxide, or titanium oxide coated synthetic mica flakes a layer consisting of from 50 to 95% by weight carbon, from 5 to 25% by weight nitrogen and from 0 to 25% by weight of the elements hydrogen, oxygen and/or sulfur, the percentage by weight data relating to the total weight of the layer (PAN).

The thickness of the nitrogen-doped carbon layer is generally from 10 to 150 nm, preferably from 30 to 70 nm. In said embodiment preferred pigments have the following layer structure:

Synthetic mica substrate/TiO₂/PAN, synthetic mica substrate/TiO₂/PAN/TiO₂, synthetic mica substrate/TiO₂/PAN/SiO₂/PAN.

In an especially preferred embodiment, the interference pigments on the basis of the synthetic mica substrate comprise a layer of a dielectric material having a “high” refractive index, that is to say a refractive index greater than about 1.65, preferably greater than about 2.0, most preferred greater than about 2.2, which is applied to the entire surface of the synthetic mica substrate. Examples of such a dielectric material are zinc sulfide (ZnS), zinc oxide (ZnO), zirconium oxide (ZrO₂), titanium dioxide (TiO₂), carbon, indium oxide (In₂O₃), indium tin oxide (ITO), tantalum pentoxide (Ta₂O₅), chromium oxide (Cr₂O₃), cerium oxide (CeO₂), yttrium oxide (Y₂O₃), europium oxide (Eu₂O₃), iron oxides such as iron(II)/iron(III) oxide (Fe₃O₄) and iron(III) oxide (Fe₂O₃), hafnium nitride (HfN), hafnium carbide (HfC), hafnium oxide (HfO₂), lanthanum oxide (La₂O₃), magnesium oxide (MgO), neodymium oxide (Nd₂O₃), praseodymium oxide (Pr₆O₁₁), samarium oxide (Sm₂O₃), antimony trioxide (Sb₂O₃), silicon monoxides (SiO), selenium trioxide (Se₂O₃), tin oxide (SnO₂), tungsten trioxide (WO₃), or combinations thereof. The dielectric material is preferably a metal oxide. It being possible for the metal oxide to be a single oxide or a mixture of oxides, with or without absorbing properties, for example, TiO₂, ZrO₂, Fe₂O₃, Fe₃O₄, Cr₂O₃ or ZnO, with TiO₂ being especially preferred.

It is possible to obtain pigments that are more intense in colour and more transparent by applying, on top of the TiO₂ layer, a metal oxide of low refractive index, such as SiO₂, Al₂O₃, AlOOH, B₂O₃ or a mixture thereof, preferably SiO₂, and optionally applying a further TiO₂ layer on top of the latter layer (EP-A-892832, EP-A-753545, WO93/08237, WO98/53011, WO9812266, WO9838254, WO99/20695, WO00/42111, and EP-A-1213330). Nonlimiting examples of suitable low index dielectric materials that can be used include silicon dioxide (SiO₂), aluminum oxide (Al₂O₃), and metal fluorides such as magnesium fluoride (MgF₂), aluminum fluoride (AlF₃), cerium fluoride (CeF₃), lanthanum fluoride (LaF₃), sodium aluminum fluorides (e.g., Na₃AlF₆ or Na₅Al₃F₁₄), neodymium fluoride (NdF₃), samarium fluoride (SmF₃), barium fluoride (BaF₂), calcium fluoride (CaF₂), lithium fluoride (LiF), combinations thereof, or any other low index material having an index of refraction of about 1.65 or less. For example, organic monomers and polymers can be utilized as low index materials, including dienes or alkenes such as acrylates (e.g., methacrylate), polymers of perfluoroalkenes, polytetrafluoroethylene (TEFLON), polymers of fluorinated ethylene propylene (FEP), parylene, p-xylene, combinations thereof, and the like. Additionally, the foregoing materials include evaporated, condensed and cross-linked transparent acrylate layers, which may be deposited by methods described in U.S. Pat. No. 5,877,895, the disclosure of which is incorporated herein by reference.

Accordingly, preferred interference pigments comprise besides (a) a metal oxide of high refractive index in addition (b) a metal oxide of low refractive index, wherein the difference of the refractive indices is at least 0.1.

Pigments on the basis of the synthetic zinc phlogopite substrates, which have been coated by a wet chemical method, in the indicated order are particularly preferred:

TiO₂, (SnO₂)TiO₂ (substrate: synthetic mica; layer: (SnO₂)TiO₂, preferably in the rutile modification), titanium suboxide, TiO₂/titanium suboxide, Fe₂O₃, Fe₃O₄, TiFe₂O₅, FeTiO₃, Cr₂O₃, ZrO₂, Sn(Sb)O₂, BiOCl, Al₂O₃, Ce₂S₃, MoS₂, Fe₂O₃.TiO₂ (substrate: synthetic mica; mixed layer of Fe₂O₃ and TiO₂), TiO₂/Fe₂O₃ (substrate: synthetic mica; first layer: TiO₂; second layer: Fe₂O₃), TiO₂/Berlin blau, TiO₂/Cr₂O₃, or TiO₂/FeTiO₃. In general the layer thickness ranges from 1 to 1000 nm, preferably from 1 to 300 nm.

In another particularly preferred embodiment the present invention relates to interference pigments containing at least three alternating layers of high and low refractive index, such as, for example, TiO₂/SiO₂/TiO₂, (SnO₂)TiO₂/SiO₂/TiO₂, TiO₂/SiO₂/TiO₂/SiO₂/TiO₂, Fe₂O₃/SiO₂/TiO₂, or TiO₂/SiO₂/Fe₂O₃.

Preferably the layer structure is as follows:
(a) a coating having a refractive index >1.65,
(b) a coating having a refractive index ≦1.65,
(c) a coating having a refractive index >1.65, and
(d) optionally an outer protective layer.

The thickness of the individual layers of high and low refractive index on the base substrate is essential for the optical properties of the pigment. The thickness of the individual layers, especially metal oxide layers, depends on the field of use and is generally 10 to 1000 nm, preferably 15 to 800 nm, in particular 20 to 600 nm.

The thickness of layer (A) is 10 to 550 nm, preferably 15 to 400 nm and, in particular, 20 to 350 nm. The thickness of layer (B) is 10 to 1000 nm, preferably 20 to 800 nm and, in particular, 30 to 600 nm. The thickness of layer (C) is 10 to 550 nm, preferably 15 to 400 nm and, in particular, 20 to 350 nm.

Particularly suitable materials for layer (A) are metal oxides, metal sulfides, or metal oxide mixtures, such as TiO₂, Fe₂O₃, TiFe₂O₅, Fe₃O₄, BiOCl, CoO, Co₃O₄, Cr₂O₃, VO₂, V₂O₃, Sn(Sb)O₂, SnO₂, ZrO₂, iron titanates, iron oxide hydrates, titanium suboxides (reduced titanium species having oxidation states from 2 to <4), bismuth vanadate, cobalt aluminate, and also mixtures or mixed phases of these compounds with one another or with other metal oxides. Metal sulfide coatings are preferably selected from sulfides of tin, silver, lanthanum, rare earth metals, preferably cerium, chromium, molybdenum, tungsten, iron, cobalt and/or nickel.

Particularly suitable materials for layer (B) are metal oxides or the corresponding oxide hydrates, such as SiO₂, MgF₂, Al₂O₃, AlOOH, B₂O₃ or a mixture thereof, preferably SiO₂.

Particularly suitable materials for layer (C) are colorless or colored metal oxides, such as TiO₂, Fe₂O₃, TiFe₂O₅, Fe₃O₄, BiOCl, CoO, Co₃O₄, Cr₂O₃, VO₂, V₂O₃, Sn(Sb)O₂, SnO₂, ZrO₂, iron titanates, iron oxide hydrates, titanium suboxides (reduced titanium species having oxidation states from 2 to <4), bismuth vanadate, cobalt aluminate, and also mixtures or mixed phases of these compounds with one another or with other metal oxides. The TiO₂ layers can additionally contain an absorbing material, such as carbon, selectively absorbing colorants, selectively absorbing metal cations, can be coated with absorbing material, or can be partially reduced.

Interlayers of absorbing or nonabsorbing materials can be present between layers (A), (B), (C) and (D). The thickness of the interlayers is 1 to 50 nm, preferably 1 to 40 nm and, in particular, 1 to 30 nm. Such an interlayer can, for example, consist of SnO₂. It is possible to force the rutile structure to be formed by adding small amounts of SnO₂ (see, for example, WO93/08237).

In this embodiment preferred interference pigments have the following layer structure:

| | | | |
|---|---|---|---|
| synthetic | TiO₂ | SiO₂ | TiO₂ |
| mica, | | | |
| especially | | | |
| Zn phlogopite | | | |
| synthetic | TiO₂ | SiO₂ | Fe₂O₃ |
| mica, Zn | | | |
| phlogopite | | | |
| synthetic | TiO₂ | SiO₂ | TiO₂•Fe₂O₃ |
| mica, Zn | | | |
| phlogopite | | | |
| synthetic | TiO₂ | SiO₂ | (Sn,Sb)O₂ |
| mica, Zn | | | |
| phlogopite | | | |
| synthetic | (Sn,Sb)O₂ | SiO₂ | TiO₂ |
| mica, Zn | | | |
| phlogopite | | | |
| synthetic | Fe₂O₃ | SiO₂ | (Sn,Sb)O₂ |
| mica, Zn | | | |
| phlogopite | | | |
| synthetic | TiO₂•Fe₂O₃ | SiO₂ | TiO₂•Fe₂O₃ |
| mica, Zn | | | |
| phlogopite | | | |
| synthetic | TiO₂ | SiO₂ | MoS₂ |
| mica, Zn | | | |
| phlogopite | | | |
| synthetic | TiO₂ | SiO₂ | Cr₂O₃ |
| mica, Zn | | | |
| phlogopite | | | |
| synthetic | Cr₂O₃ | SiO₂ | TiO₂ |
| mica, Zn | | | |
| phlogopite | | | |
| synthetic | Fe₂O₃ | SiO₂ | TiO₂ |
| mica, Zn | | | |
| phlogopite | | | |
| Synthetic | Fe₂O₃ | Al₂O₃ | Fe₂O₃ |
| mica, Zn | | | |
| phlogopite | | | |
| Synthetic | TiO₂ | Al₂O₃ | Fe₂O₃ |
| mica, Zn | | | |
| phlogopite | | | |
| Synthetic | Fe₂O₃ | SiO₂ | Fe₂O₃ |
| mica, Zn | | | |
| phlogopite | | | |
| synthetic | TiO₂ | Al₂O₃ | TiO₂ |
| mica, Zn | | | |
| phlogopite | | | |
| synthetic | Fe₂TiO₅ | SiO₂ | TiO₂ |
| mica, Zn | | | |
| phlogopite | | | |
| synthetic | TiO₂ | SiO₂ | Fe₂TiO₅/ |
| mica, Zn | | | TiO₂ |
| phlogopite | | | |
| synthetic | TiO | SiO₂ | TiO |
| mica, Zn | suboxides | | suboxides |
| phlogopite | | | |
| synthetic | TiO₂ | SiO₂ | TiO₂/SiO₂/ |
| mica, Zn | | | TiO₂ + Prussian Blue |
| phlogopite | | | |
| synthetic | TiO₂ | SiO₂ | TiO₂/SiO₂/ |
| mica, Zn | | | TiO₂ |
| phlogopite | | | |
| synthetic | TiO₂/SiO₂/ | SiO₂ | TiO₂/SiO₂/ |
| mica, Zn | TiO₂ | | TiO₂ |
| phlogopite * | | | |

* Zn phlogopite characterized by an diameter of at least 2.5 microns, preferably a diameter of at least 3.4 and most preferably a diameter of at least 5.

The metal oxide layers can be applied by CVD (chemical vapour deposition) or by wet chemical coating. The metal oxide layers can be obtained by decomposition of metal carbonyls in the presence of water vapour (relatively low molecular weight metal oxides such as magnetite) or in the presence of oxygen and, where appropriate, water vapour (e.g. nickel oxide and cobalt oxide). The metal oxide layers are especially applied by means of oxidative gaseous phase decomposition of metal carbonyls (e.g. iron pentacarbonyl, chromium hexacarbonyl; EP-A-45 851), by means of hydrolytic gaseous phase decomposition of metal alcoholates (e.g. titanium and zirconium tetra-n- and -iso-propanolate; DE-A-41 40 900) or of metal halides (e.g. titanium tetrachloride; EP-A-338 428), by means of oxidative decomposition of organyl tin compounds (especially alkyl tin compounds such as tetrabutyltin and tetramethyltin; DE-A-44 03 678) or by means of the gaseous phase hydrolysis of organyl silicon compounds (especially di-tert-butoxyacetoxysilane) described in EP-A-668 329, it being possible for the coating operation to be carried out in a fluidised-bed reactor (EP-A-045 851 and EP-A-106 235). Al₂O₃ layers (B) can advantageously be obtained by controlled oxidation during the cooling of aluminium-coated pigments, which is otherwise carried out under inert gas (DE-A-195 16 181).

Phosphate-, chromate- and/or vanadate-containing and also phosphate- and SiO₂-containing metal oxide layers can be applied in accordance with the passivation methods described in DE-A-42 36 332 and in EP-A-678 561 by means of hydrolytic or oxidative gaseous phase decomposition of oxide-halides of the metals (e.g. CrO₂Cl₂, VOCl₃), especially of phosphorus oxyhalides (e.g. POCl₃), phosphoric and phosphorous acid esters (e.g. di- and tri-methyl and di- and tri-ethyl phosphite) and of amino-group-containing organyl silicon compounds (e.g. 3-aminopropyl-triethoxy- and -trimethoxy-silane).

Layers of oxides of the metals zirconium, titanium, iron and zinc, oxide hydrates of those metals, iron titanates, titanium suboxides or mixtures thereof are preferably applied by precipitation by a wet chemical method, it being possible, where appropriate, for the metal oxides to be reduced. In the case of the wet chemical coating, the wet chemical coating methods developed for the production of pearlescent pigments may be used; these are described, for example, in DE-A-14 67 468, DE-A-19 59 988, DE-A-20 09 566, DE-A-22 14 545, DE-A-22 15 191, DE-A-22 44 298, DE-A-23 13 331, DE-A-25 22 572, DE-A-31 37 808, DE-A-31 37 809, DE-A-31 51 343, DE-A-31 51 354, DE-A-31 51 355, DE-A-32 11 602 and DE-A-32 35 017, DE 195 99 88, WO 93/08237, WO 98/53001 and WO03/6558.

The metal oxide of high refractive index is preferably TiO₂ and/or iron oxide, and the metal oxide of low refractive index is preferably SiO₂. Layers of TiO₂ can be in the rutile or anastase modification, wherein the rutile modification is preferred. TiO₂ layers can also be reduced by known means, for example ammonia, hydrogen, hydrocarbon vapor or mixtures thereof, or metal powders, as described in EP-A-735,114, DE-A-3433657, DE-A-4125134, EP-A-332071, EP-A-707,050, WO93/19131, or WO06/131472.

For the purpose of coating, the substrate particles are suspended in water and one or more hydrolysable metal salts are added at a pH suitable for the hydrolysis, which is so selected that the metal oxides or metal oxide hydrates are precipitated directly onto the particles without subsidiary precipitation occurring. The pH is usually kept constant by simultaneously metering in a base. The pigments are then separated off, washed, dried and, where appropriate, calcinated, it being possible to optimise the calcinating temperature with respect to the coating in question. If desired, after individual coatings have been applied, the pigments can be separated off, dried and, where appropriate, calcinated, and then again re-suspended for the purpose of precipitating further layers.

The metal oxide layers are also obtainable, for example, in analogy to a method described in DE-A-195 01 307, by producing the metal oxide layer by controlled hydrolysis of one or more metal acid esters, where appropriate in the presence of an organic solvent and a basic catalyst, by means of a sol-gel process. Suitable basic catalysts are, for example, amines, such as triethylamine, ethylenediamine, tributylamine, dimethylethanolamine and methoxy-propylamine. The organic solvent is a water-miscible organic solvent such as a C₁₋₄alcohol, especially isopropanol.

Suitable metal acid esters are selected from alkyl and aryl alcoholates, carboxylates, and carboxyl-radical- or alkyl-radical- or aryl-radical-substituted alkyl alcoholates or carboxylates of vanadium, titanium, zirconium, silicon, aluminium and boron. The use of triisopropyl aluminate, tetraisopropyl titanate, tetraisopropyl zirconate, tetraethyl orthosilicate and triethyl borate is preferred. In addition, acetylacetonates and acetoacetylacetonates of the afore-mentioned metals may be used. Preferred examples of that type of metal acid ester are zirconium acetylacetonate, aluminium acetylacetonate, titanium acetylacetonate and diisobutyloleyl acetoacetylaluminate or diisopropyloleyl acetoacetylacetonate and mixtures of metal acid esters, for example Dynasil® (Hüls), a mixed aluminium/silicon metal acid ester.

As a metal oxide having a high refractive index, titanium dioxide is preferably used, the method described in U.S. Pat. No. 3,553,001 being used, in accordance with an embodiment of the present invention, for application of the titanium dioxide layers.

An aqueous titanium salt solution is slowly added to a suspension of the material being coated, which suspension has been heated to about 50-100° C., especially 70-80° C., and a substantially constant pH value of about from 0.5 to 5, especially about from 1.2 to 2.5, is maintained by simultaneously metering in a base such as, for example, aqueous ammonia solution or aqueous alkali metal hydroxide solution. As soon as the desired layer thickness of precipitated TiO₂ has been achieved, the addition of titanium salt solution and base is stopped. Addition of a precursor for Al₂O₃ or MgO in the starting solutions is a way for improving the morphology of the TiO₂ layer.

This method, also referred to as the “titration method”, is distinguished by the fact that an excess of titanium salt is avoided. That is achieved by feeding in for hydrolysis, per unit time, only that amount which is necessary for even coating with the hydrated TiO₂ and which can be taken up per unit time by the available surface of the particles being coated. In principle, the anatase form of TiO₂ forms on the surface of the starting pigment. By adding small amounts of SnO₂, however, it is possible to force the rutile structure to be formed. For example, as described in WO 93/08237, tin dioxide can be deposited before titanium dioxide precipitation and the product coated with titanium dioxide can be calcined at from 800 to 900° C.

In an especially preferred embodiment of the present invention the synthetic mica flakes are mixed with distilled water in a closed reactor and heated at about 90° C. The pH is set to about 1.8 to 2.2 and a preparation comprising TiOCl₂, HCl, glycine and distilled water is added slowly while keeping the pH constant (1.8 to 2.2) by continuous addition of 1M NaOH solution. Reference is made to European patent application PCT/EP2008/051910. By adding an amino acid, such as glycine, during the deposition of the TiO₂ it is possible to improve the quality of the TiO₂ coating to be formed. Advantageously, a preparation comprising TiOCl₂, HCl, and glycine and distilled water is added to the substrate flakes in water.

The TiO₂ can optionally be reduced by usual procedures: U.S. Pat. No. 4,948,631 (NH₃, 750-850° C.), WO93/19131 (H₂, >900° C.) or DE-A-19843014 (solid reduction agent, such as, for example, silicon, >600° C.).

Where appropriate, an SiO₂ (protective) layer can be applied on top of the titanium dioxide layer, for which the following method may be used: A soda waterglass solution is metered into a suspension of the material being coated, which suspension has been heated to about 50-100° C., especially 70-80° C. The pH is maintained at from 4 to 10, preferably from 6.5 to 8.5, by simultaneously adding 10% hydrochloric acid. After addition of the waterglass solution, stirring is carried out for 30 minutes.

It is possible to obtain pigments that are more intense in colour and more transparent by applying, on top of the TiO₂ layer, a metal oxide of “low” refractive index, that is to say a refractive index smaller than about 1.65, such as SiO₂, Al₂O₃, AlOOH, B₂O₃ or a mixture thereof, preferably SiO₂, and applying a further Fe₂O₃ and/or TiO₂ layer on top of the latter layer. Such multi-coated interference pigments comprising a synthetic mica substrate and alternating metal oxide layers of with high and low refractive index can be prepared in analogy to the processes described in WO98/53011 and WO99/20695.

It is, in addition, possible to modify the powder colour of the pigment by applying further layers such as, for example, coloured metal oxides or Berlin Blue, compounds of transition metals, e.g. Fe, Cu, Ni, Co, Cr, or organic compounds such as dyes or colour lakes.

In addition, the pigment according to the invention can also be coated with poorly soluble, firmly adhering, inorganic or organic colourants. Preference is given to the use of colour lakes and, especially, aluminium colour lakes. For that purpose an aluminium hydroxide layer is precipitated, which is, in a second step, laked by using a colour lake (DE-A-24 29 762 and DE-A-29 28 287).

Furthermore, the pigment according to the invention may also have an additional coating with complex salt pigments, especially cyanoferrate complexes (EP-A-141 173 and DE-A-23 13 332).

To enhance the weather and light stability the (multilayer) synthetic zinc phlogopite flakes can be, depending on the field of application, subjected to a surface treatment. Useful surface treatments are, for example, described in DE-A-2215191, DE-A-3151354, DE-A-3235017, DE-A-3334598, DE-A-4030727, EP-A-649886, WO97/29059, WO99/57204, and U.S. Pat. No. 5,759,255. Said surface treatment might also facilitate the handling of the pigment, especially its incorporation into various application media.

In a preferred embodiment of the present invention is directed to pigments which contain a core of synthetic mica and comprise a mixed layer of Al₂O₃/TiO₂. The mixed layer can contain up to 20 mol % Al₂O₃. The mixed layer of Al₂O₃/TiO₂ is obtained by slowly adding an aqueous aluminum and titanium salt solution to a suspension of the material being coated, which suspension has been heated to about 50-100° C., especially 70-80° C., and maintaining a substantially constant pH value of about from 0.5 to 5, especially about from 1.2 to 2.5, by simultaneously metering in a base such as, for example, aqueous ammonia solution or aqueous alkali metal hydroxide solution. As soon as the desired layer thickness of precipitated Al₂O₃/TiO₂ has been achieved, the addition of titanium and aluminum salt solution and base is stopped.

The thickness of the mixed layer of Al₂O₃/TiO₂ is in general in the range of 20 to 200 nm, especially 50 to 150 nm. Preferably the pigments comprise a TiO₂ layer on top of the mixed layer of Al₂O₃/TiO₂ having a thickness of 1 to 50 nm, especially 10 to 20 nm. By varying the thickness of the mixed layer of Al₂O₃/TiO₂ the flop of the pigments can be enhanced and controlled as desired.

In another preferred embodiment of the present invention is directed to pigments which contain a core of the zinc phlogopite of diameter 2 microns or greater and consist of subsequent layers of TiO₂/SnO₂/TiO₂, wherein the TiO₂ layer next to the synthetic mica substrate has a thickness of 1 to 20 nm and is preferably prepared by using titanium alcoholates, especially tetraisopropyl titanate.

Metallic or non-metallic, inorganic platelet-shaped particles or pigments are effect pigments, (especially metal effect pigments or interference pigments), that is to say, pigments that, besides imparting colour to an application medium, impart additional properties, for example angle dependency of the colour (flop), lustre (not surface gloss) or texture. On metal effect pigments, substantially oriented reflection occurs at directionally oriented pigment particles. In the case of interference pigments, the colour-imparting effect is due to the phenomenon of interference of light in thin, highly refractive layers.

The (effect) pigments according to the invention can be used for all customary purposes, for example for colouring polymers in the mass, coatings (including effect finishes, including those for the automotive sector) and printing inks (including offset printing, intaglio printing, bronzing and flexographic printing), and also, for example, for applications in cosmetics, in ink-jet printing, for dyeing textiles, glazes for ceramics and glass as well as laser marking of papers and plastics. Such applications are known from reference works, for example “Industrielle Organische Pigmente” (W. Herbst and K. Hunger, VCH Verlagsgesellschaft mbH, Weinheim/New York, 2nd, completely revised edition, 1995).

When the pigments according to the invention are interference pigments (effect pigments), they may be goniochromatic and result in brilliant, highly saturated (lustrous) colours. They are accordingly very especially suitable for combination with conventional, transparent pigments, for example organic pigments such as, for example, diketopyrrolopyrroles, quinacridones, dioxazines, perylenes, isoindolinones etc., it being possible for the transparent pigment to have a similar colour to the effect pigment. Especially interesting combination effects are obtained, however, in analogy to, for example, EP-A-388 932 or EP-A-402 943, when the colour of the transparent pigment and that of the effect pigment are complementary. The pigments according to the invention can be used with excellent results for pigmenting high molecular weight organic material.

The high molecular weight organic material for the pigmenting of which the pigments or pigment compositions according to the invention may be used may be of natural or synthetic origin. High molecular weight organic materials usually have average weight average molecular weights of about from 10³ to 10⁸ g/mol or even more. They may be, for example, natural resins, drying oils, rubber or casein, or natural substances derived therefrom, such as chlorinated rubber, oil-modified alkyd resins, viscose, cellulose ethers or esters, such as ethylcellulose, cellulose acetate, cellulose propionate, cellulose acetobutyrate or nitrocellulose, but especially totally synthetic organic polymers (thermosetting plastics and thermoplastics), as are obtained by polymerisation, polycondensation or polyaddition. From the class of the polymerisation resins there may be mentioned, especially, polyolefins, such as polyethylene, polypropylene or polyisobutylene, and also substituted polyolefins, such as polymerisation products of vinyl chloride, vinyl acetate, styrene, acrylonitrile, acrylic acid esters, methacrylic acid esters or butadiene, and also copolymerisation products of the said monomers, such as especially ABS or EVA.

From the series of the polyaddition resins and polycondensation resins there may be mentioned, for example, condensation products of formaldehyde with phenols, so-called phenoplasts, and condensation products of formaldehyde with urea, thiourea or melamine, so-called aminoplasts, and the polyesters used as surface-coating resins, either saturated, such as alkyd resins, or unsaturated, such as maleate resins; also linear polyesters and polyamides, polyurethanes or silicones.

The said high molecular weight compounds may be present singly or in mixtures, in the form of plastic masses or melts. They may also be present in the form of their monomers or in the polymerised state in dissolved form as film-formers or binders for coatings or printing inks, such as, for example, boiled linseed oil, nitrocellulose, alkyd resins, melamine resins and urea-formaldehyde resins or acrylic resins.

Depending on the intended purpose, it has proved advantageous to use the effect pigments or effect pigment compositions according to the invention as toners or in the form of preparations. Depending on the conditioning method or intended application, it may be advantageous to add certain amounts of texture-improving agents to the effect pigment before or after the conditioning process, provided that this has no adverse effect on use of the effect pigments for colouring high molecular weight organic materials, especially polyethylene. Suitable agents are, especially, fatty acids containing at least 18 carbon atoms, for example stearic or behenic acid, or amides or metal salts thereof, especially magnesium salts, and also plasticisers, waxes, resin acids, such as abietic acid, rosin soap, alkylphenols or aliphatic alcohols, such as stearyl alcohol, or aliphatic 1,2-dihydroxy compounds containing from 8 to 22 carbon atoms, such as 1,2-dodecanediol, and also modified colophonium maleate resins or fumaric acid colophonium resins. The texture-improving agents are added in amounts of preferably from 0.1 to 30% by weight, especially from 2 to 15% by weight, based on the end product.

The (effect) pigments according to the invention can be added in any tinctorially effective amount to the high molecular weight organic material being pigmented. A pigmented substance composition comprising a high molecular weight organic material and from 0.01 to 80% by weight, preferably from 0.1 to 30% by weight, based on the high molecular weight organic material, of an pigment according to the invention is advantageous. Concentrations of from 1 to 20% by weight, especially of about 10% by weight, can often be used in practice.

High concentrations, for example those above 30% by weight, are usually in the form of concentrates (“masterbatches”) which can be used as colorants for producing pigmented materials having a relatively low pigment content, the pigments according to the invention having an extraordinarily low viscosity in customary formulations so that they can still be processed well.

For the purpose of pigmenting organic materials, the effect pigments according to the invention may be used singly. It is, however, also possible, in order to achieve different hues or colour effects, to add any desired amounts of other colour-imparting constituents, such as white, coloured, black or effect pigments, to the high molecular weight organic substances in addition to the effect pigments according to the invention. When coloured pigments are used in admixture with the effect pigments according to the invention, the total amount is preferably from 0.1 to 10% by weight, based on the high molecular weight organic material. Especially high goniochromicity is provided by the preferred combination of an effect pigment according to the invention with a coloured pigment of another colour, especially of a complementary colour, with colorations made using the effect pigment and colorations made using the coloured pigment having, at a measurement angle of 10°, a difference in hue (ΔH*) of from 20 to 340, especially from 150 to 210.

Preferably, the effect pigments according to the invention are combined with transparent coloured pigments, it being possible for the transparent coloured pigments to be present either in the same medium as the effect pigments according to the invention or in a neighbouring medium. An example of an arrangement in which the effect pigment and the coloured pigment are advantageously present in neighbouring media is a multi-layer effect coating.

The pigmenting of high molecular weight organic substances with the pigments according to the invention is carried out, for example, by admixing such a pigment, where appropriate in the form of a masterbatch, with the substrates using roll mills or mixing or grinding apparatuses. The pigmented material is then brought into the desired final form using methods known per se, such as calendering, compression moulding, extrusion, coating, pouring or injection moulding. Any additives customary in the plastics industry, such as plasticisers, fillers or stabilisers, can be added to the polymer, in customary amounts, before or after incorporation of the pigment. In particular, in order to produce non-rigid shaped articles or to reduce their brittleness, it is desirable to add plasticisers, for example esters of phosphoric acid, phthalic acid or sebacic acid, to the high molecular weight compounds prior to shaping.

For pigmenting coatings and printing inks, the high molecular weight organic materials and the effect pigments according to the invention, where appropriate together with customary additives such as, for example, fillers, other pigments, siccatives or plasticisers, are finely dispersed or dissolved in the same organic solvent or solvent mixture, it being possible for the individual components to be dissolved or dispersed separately or for a number of components to be dissolved or dispersed together, and only thereafter for all the components to be brought together.

Dispersing an effect pigment according to the invention in the high molecular weight organic material being pigmented, and processing a pigment composition according to the invention, are preferably carried out subject to conditions under which only relatively weak shear forces occur so that the effect pigment is not broken up into smaller portions.

Plastics comprising the pigment of the invention in amounts of 0.1 to 50% by weight, in particular 0.5 to 7% by weight. In the coating sector, the pigments of the invention are employed in amounts of 0.1 to 10% by weight. In the pigmentation of binder systems, for example for paints and printing inks for intaglio, offset or screen printing, the pigment is incorporated into the printing ink in amounts of 0.1 to 50% by weight, preferably 5 to 30% by weight and in particular 8 to 15% by weight.

The colorations obtained, for example in plastics, coatings or printing inks, especially in coatings or printing inks, more especially in coatings, may be distinguished by excellent properties, especially by extremely high saturation, outstanding fastness properties, high color purity and high goniochromaticity.

When the high molecular weight material being pigmented is a coating, it is especially a specialty coating, very especially an automotive finish.

The effect pigments according to the invention are also suitable for making-up the lips or the skin and for colouring the hair or the nails.

The invention accordingly relates also to a cosmetic preparation or formulation comprising from 0.0001 to 90% by weight of a pigment, especially an effect pigment, according to the invention and from 10 to 99.9999% of a cosmetically suitable carrier material, based on the total weight of the cosmetic preparation or formulation.

Such cosmetic preparations or formulations are, for example, lipsticks, blushers, foundations, nail varnishes and hair shampoos.

The pigments may be used singly or in the form of mixtures. It is, in addition, possible to use pigments according to the invention together with other pigments and/or colorants, for example in combinations as described hereinbefore or as known in cosmetic preparations.

The cosmetic preparations and formulations according to the invention preferably contain the pigment according to the invention in an amount from 0.005 to 50% by weight, based on the total weight of the preparation.

Suitable carrier materials for the cosmetic preparations and formulations according to the invention include the customary materials used in such compositions.

The cosmetic preparations and formulations according to the invention may be in the form of, for example, sticks, ointments, creams, emulsions, suspensions, dispersions, powders or solutions. They are, for example, lipsticks, mascara preparations, blushers, eye-shadows, foundations, eyeliners, powder or nail varnishes.

If the preparations are in the form of sticks, for example lipsticks, eye-shadows, blushers or foundations, the preparations consist for a considerable part of fatty components, which may consist of one or more waxes, for example ozokerite, lanolin, lanolin alcohol, hydrogenated lanolin, acetylated lanolin, lanolin wax, beeswax, candelilla wax, microcrystalline wax, carnauba wax, cetyl alcohol, stearyl alcohol, cocoa butter, lanolin fatty acids, petrolatum, petroleum jelly, mono-, di- or tri-glycerides or fatty esters thereof that are solid at 25° C., silicone waxes, such as methyloctadecane-oxypolysiloxane and poly(dimethylsiloxy)stearoxysiloxane, stearic acid monoethanolamine, colophane and derivatives thereof, such as glycol abietates and glycerol abietates, hydrogenated oils that are solid at 25° C., sugar glycerides and oleates, myristates, lanolates, stearates and dihydroxystearates of calcium, magnesium, zirconium and aluminium.

The fatty component may also consist of a mixture of at least one wax and at least one oil, in which case the following oils, for example, are suitable: paraffin oil, purcelline oil, perhydrosqualene, sweet almond oil, avocado oil, calophyllum oil, castor oil, sesame oil, jojoba oil, mineral oils having a boiling point of about from 310 to 410° C., silicone oils, such as dimethylpolysiloxane, linoleyl alcohol, linolenyl alcohol, ° leyl alcohol, cereal grain oils, such as wheatgerm oil, isopropyl lanolate, isopropyl palmitate, isopropyl myristate, butyl myristate, cetyl myristate, hexadecyl stearate, butyl stearate, decyl oleate, acetyl glycerides, octanoates and decanoates of alcohols and polyalcohols, for example of glycol and glycerol, ricinoleates of alcohols and polyalcohols, for example of cetyl alcohol, isostearyl alcohol, isocetyl lanolate, isopropyl adipate, hexyl laurate and octyl dodecanol.

The fatty components in such preparations in the form of sticks may generally constitute up to 99.91% by weight of the total weight of the preparation.

The cosmetic preparations and formulations according to the invention may additionally comprise further constituents, such as, for example, glycols, polyethylene glycols, polypropylene glycols, monoalkanolamides, non-coloured polymeric, inorganic or organic fillers, preservatives, UV filters or other adjuvants and additives customary in cosmetics, for example a natural or synthetic or partially synthetic di- or tri-glyceride, a mineral oil, a silicone oil, a wax, a fatty alcohol, a Guerbet alcohol or ester thereof, a lipophilic functional cosmetic active ingredient, including sun-protection filters, or a mixture of such substances.

A lipophilic functional cosmetic active ingredient suitable for skin cosmetics, an active ingredient composition or an active ingredient extract is an ingredient or a mixture of ingredients that is approved for dermal or topical application. The following may be mentioned by way of example:
active ingredients having a cleansing action on the skin surface and the hair; these include all substances that serve to cleanse the skin, such as oils, soaps, synthetic detergents and solid substances;
active ingredients having a deodorising and perspiration-inhibiting action: they include antiperspirants based on aluminium salts or zinc salts, deodorants comprising bactericidal or bacteriostatic deodorising substances, for example triclosan, hexachlorophene, alcohols and cationic substances, such as, for example, quaternary ammonium salts, and odour absorbers, for example ®Grillocin (combination of zinc ricinoleate and various additives) or triethyl citrate (optionally in combination with an antioxidant, such as, for example, butyl hydroxytoluene) or ion-exchange resins;
active ingredients that offer protection against sunlight (UV filters): suitable active ingredients are filter substances (sunscreens) that are able to absorb UV radiation from sunlight and convert it into heat; depending on the desired action, the following light-protection agents are preferred: light-protection agents that selectively absorb sunburn-causing high-energy UV radiation in the range of approximately from 280 to 315 nm (UV-B absorbers) and transmit the longer-wavelength range of, for example, from 315 to 400 nm (UV-A range), as well as light-protection agents that absorb only the longer-wavelength radiation of the UV-A range of from 315 to 400 nm (UV-A absorbers); suitable light-protection agents are, for example, organic UV absorbers from the class of the p-aminobenzoic acid derivatives, salicylic acid derivatives, benzophenone derivatives, dibenzoylmethane derivatives, diphenyl acrylate derivatives, benzofuran derivatives, polymeric UV absorbers comprising one or more organosilicon radicals, cinnamic acid derivatives, camphor derivatives, trianilino-s-triazine derivatives, phenyl-benzimidazolesulfonic acid and salts thereof, menthyl anthranilates, benzotriazole derivatives, and/or an inorganic micropigment selected from aluminium oxide- or silicon dioxide-coated TiO₂, zinc oxide or mica;
active ingredients against insects (repellents) are agents that are intended to prevent insects from touching the skin and becoming active there; they drive insects away and evaporate slowly; the most frequently used repellent is diethyl toluamide (DEET); other common repellents will be found, for example, in “Pflegekosmetik” (W. Raab and U. Kindl, Gustav-Fischer-Verlag Stuttgart/New York, 1991) on page 161;
active ingredients for protection against chemical and mechanical influences: these include all substances that form a barrier between the skin and external harmful substances, such as, for example, paraffin oils, silicone oils, vegetable oils, PCL products and lanolin for protection against aqueous solutions, film-forming agents, such as sodium alginate, triethanolamine alginate, polyacrylates, polyvinyl alcohol or cellulose ethers for protection against the effect of organic solvents, or substances based on mineral oils, vegetable oils or silicone oils as “lubricants” for protection against severe mechanical stresses on the skin;
moisturising substances: the following substances, for example, are used as moisture-controlling agents (moisturisers): sodium lactate, urea, alcohols, sorbitol, glycerol, propylene glycol, collagen, elastin and hyaluronic acid;
active ingredients having a keratoplastic effect: benzoyl peroxide, retinoic acid, colloidal sulfur and resorcinol;
antimicrobial agents, such as, for example, triclosan or quaternary ammonium compounds;
oily or oil-soluble vitamins or vitamin derivatives that can be applied dermally: for example vitamin A (retinol in the form of the free acid or derivatives thereof), panthenol, pantothenic acid, folic acid, and combinations thereof, vitamin E (tocopherol), vitamin F; essential fatty acids; or niacinamide (nicotinic acid amide);
vitamin-based placenta extracts: active ingredient compositions comprising especially vitamins A, C, E, B₁, B₂, B₆, B₁₂, folic acid and biotin, amino acids and enzymes as well as compounds of the trace elements magnesium, silicon, phosphorus, calcium, manganese, iron or copper;
skin repair complexes: obtainable from inactivated and disintegrated cultures of bacteria of the bifidus group;
plants and plant extracts: for example arnica, aloe, beard lichen, ivy, stinging nettle, ginseng, henna, camomile, marigold, rosemary, sage, horsetail or thyme;
animal extracts: for example royal jelly, propolis, proteins or thymus extracts;
cosmetic oils that can be applied dermally: neutral oils of the Miglyol 812 type, apricot kernel oil, avocado oil, babassu oil, cottonseed oil, borage oil, thistle oil, groundnut oil, gamma-oryzanol, rosehip-seed oil, hemp oil, hazelnut oil, blackcurrant-seed oil, jojoba oil, cherry-stone oil, salmon oil, linseed oil, cornseed oil, macadamia nut oil, almond oil, evening primrose oil, mink oil, olive oil, pecan nut oil, peach kernel oil, pistachio nut oil, rape oil, rice-seed oil, castor oil, safflower oil, sesame oil, soybean oil, sunflower oil, tea tree oil, grapeseed oil or wheatgerm oil.

The preparations in stick form are preferably anhydrous but may in certain cases comprise a certain amount of water which, however, in general does not exceed 40% by weight, based on the total weight of the cosmetic preparation.

If the cosmetic preparations and formulations according to the invention are in the form of semi-solid products, that is to say in the form of ointments or creams, they may likewise be anhydrous or aqueous. Such preparations and formulations are, for example, mascaras, eyeliners, foundations, blushers, eye-shadows, or compositions for treating rings under the eyes.

If, on the other hand, such ointments or creams are aqueous, they are especially emulsions of the water-in-oil type or of the oil-in-water type that comprise, apart from the pigment, from 1 to 98.8% by weight of the fatty phase, from 1 to 98.8% by weight of the aqueous phase and from 0.2 to 30% by weight of an emulsifier.

Such ointments and creams may also comprise further conventional additives, such as, for example, perfumes, antioxidants, preservatives, gel-forming agents, UV filters, colorants, pigments, pearlescent agents, non-coloured polymers as well as inorganic or organic fillers. If the preparations are in the form of a powder, they consist substantially of a mineral or inorganic or organic filler such as, for example, talcum, kaolin, starch, polyethylene powder or polyamide powder, as well as adjuvants such as binders, colorants etc.

Such preparations may likewise comprise various adjuvants conventionally employed in cosmetics, such as fragrances, antioxidants, preservatives etc.

If the cosmetic preparations and formulations according to the invention are nail varnishes, they consist essentially of nitrocellulose and a natural or synthetic polymer in the form of a solution in a solvent system, it being possible for the solution to comprise other adjuvants, for example pearlescent agents.

In that embodiment, the coloured polymer is present in an amount of approximately from 0.1 to 5% by weight.

The cosmetic preparations and formulations according to the invention may also be used for colouring the hair, in which case they are used in the form of shampoos, creams or gels that are composed of the base substances conventionally employed in the cosmetics industry and a pigment according to the invention.

The cosmetic preparations and formulations according to the invention are prepared in conventional manner, for example by mixing or stirring the components together, optionally with heating so that the mixtures melt.

Thus the present application envisions cosmetics, coatings, inks, paints, and plastic composition containing the effect pigment formed from a coated zinc phlogopite of a diameter of 2 microns or greater.

Barrier Applications of the Hydrothermally Produced Mica

The synthetically derived zinc phlogopite platelets produced via the hydrothermal process above may be used to effect barrier to gases such as water vapor, CO₂ and oxygen barriers when present in paper coatings, coatings on packaging films or melt blended in films or containers used in packaging.

The platelets formed by the presently disclosed process may be used to form layered structures on or in such substrates such as paper, plastic packaging or as component within a coating. The layered structures of mica materials, for example, may be used to provide a barrier packaging film with a low moisture vapor transmission rate (MVTR), and/or a low oxygen transmission rate (OTR).

It is well known to use layered silicates to improve the flame retardant properties of flammable substrates. For example the zinc phlogopite platelet formed by the present hydrothermal process, may be used in polymeric composites for improving the flame retardant properties of the composite by increasing the barrier properties of the composite, and increased char formation upon ignition of the composite.

### EXAMPLES

### Example 1

The starting reagents are Al(NO₃)₃, zinc sulfate heptahydrate, potassium hydroxide, and colloidal silica. A 6M KOH solution is added to zinc sulfate heptahydrate and the mixture is stirred for approximately five minutes. 6M KOH is added to Al(NO₃)₃ and the contents are swirled to mix. The two mixtures are combined. The reaction mixture is transferred to a stirred Parr reactor and the colloidal silica is added forming a gel. The reactor is heated for 8 hours to reach a temperature of 200° C. and kept at temperature for 24 hours. Upon cooling to room temperature, the reaction is filtered and washed with D.I. water yielding a white powder. The reactants are added at non-stoichiometric ratios. This preparation follows the preparation in Komarneni et. al, Clays and Clay Minerals, Vol. 51, #6, 693, 2003 except that colloidal silica replaces H₂SiO₃ as the silicon source. The size distribution is d(10): 1.5 μm d(50): 5.5 μm d(90): 16.7 μm.

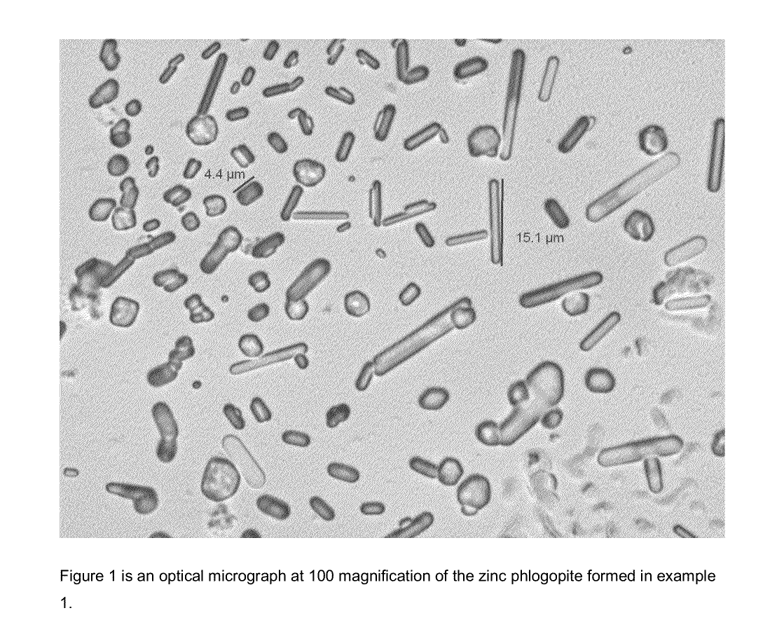

FIG. 1 shows the morphology of the zinc phlogophite formed in example 1.

### Example 2

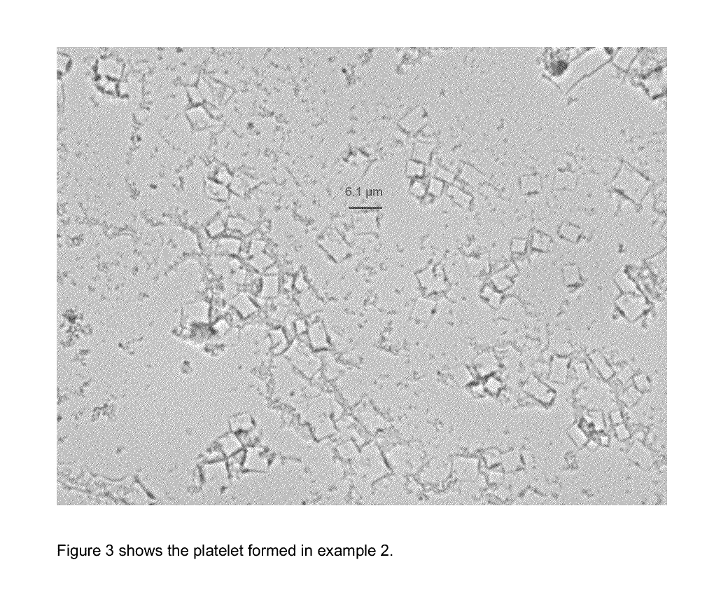

Example 2 uses aluminum nitrate as the aluminum source, is run under static mixing conditions and colloidal silica is the silicon source. The reactants are added at stoichiometric ratios. FIG. 3 shows the morphology of the platelets formed in example 2. The morphology of these platelets are very different than the morphology shown in FIG. 1. The size distribution of the particles is d(10): 0.8 μm, d(50): 2.9 μm, d(90): 27.1 μm.

### Synthesis of Zinc Phlogopite KZn₃AlSi₃O₁₀(OH)₂

TABLE I
Reactants and Reaction Conditions for Examples 1-4

| | | | Dura- | | | Stir- |
|---|---|---|---|---|---|---|
| | Gel Composition Molar | Temp | tion | % | Major Phase from | Rate |
| Example | Ratio | (° C.) | (hrs) | solids | PXRD | (rpm) |
| 1 | 46KOH, | 200 | 24 | 21 | KZn₃AlSi₃O₁₀(OH)₂ | N/A |
| | 1Al(NO₃)₃•9H₂O, 5SiO₂, | | | | | |
| | 3.71ZnSO₄•7H₂O, 956 | | | | | |
| | H₂O | | | | | |
| 2 | 28KOH, | 200 | 24 | 24 | KZn₃AlSi₃O₁₀(OH)₂ | N/A |
| | 1Al(NO₃)₃•9H₂O, 3SiO₂, | | | | | |
| | 3ZnSO₄•7H₂O, 513 H₂O | | | | | |

### Example 3

The platelets formed by the present hydrothermal method are coated with TiO₂ according the well known methods of the prior art to form an effect pigment.

1. A synthetic zinc phlogopite platelet of formula (1)
IZn₃(AlSi₃O₁₀)(X)₂ (1)
wherein
I is an interlayer monovalent cation selected from the group consisting of K⁺, Na⁺, NH₄⁺ and Li⁺;
and
X is independently fluoride, hydroxide or fluoride and hydroxide,
characterized by a diameter of at least 2.5 microns.

2. The platelet according to claim 1, wherein the
formula (1) is selected from any one of or a mixture of phlogopite formulae selected from the group consisting of KZn₃AlSi₃O₁₀(OH)₂, KZn₃AlSi₃O₁₀(OH,F) and KZn₃AlSi₃O₁₀(F), preferably KZn₃AlSi₃O₁₀(OH)₂.

3. The platelet according to claim 1, wherein the diameter d₅₀ ranges from at least 2.5 microns to about 1 mm.

4. The platelet according to claim 1, wherein thickness of the zinc phlogopite platelet will range from about 10 nm to about 500 nm.

5. The platelet according to claim 1, wherein the platelet transmits at least 92% light.

6. A process of preparing a phlogopite platelet of formula (1),
IZn₃(AlSi₃O₁₀)(X)₂ (1)
wherein
I is an interlayer monovalent cation selected from the group consisting of K⁺, Na⁺, NH₄⁺ and Li⁺;
and
X is independently fluoride, hydroxide or hydroxide and fluororine;
comprising the steps of:
forming a reaction mixture comprising
an I source selected from the group consisting of Na⁺, K⁺, NH₄⁺ and Li⁺;
an aluminum source;
a silicon source;
a zinc source;
optionally a fluoride source and/or an hydroxide source;
and
optionally seed crystals of a preformed phlogopite crystals,
hydrothermally treating said reaction mixture under basic conditions at a temperature ranging from about 125 to about 250° C.,
and
a pressure ranging from about 50 to about 400 psi;
to form the platelet of formula (1);
and
optionally isolating the formed platelet and the silicon source is colloidal silica.

7. The process according to claim 6, wherein the aluminum source, silica source, zinc source are present in the reaction mixture in stoichiometric amounts.

8. The process according to claim 6,
wherein the aluminum source is selected from the group consisting of elemental aluminum Al⁰, Al(NO₃)₃ (aluminum nitrate), K₂Al₂O₄ (potassium aluminate), Na₂Al₂O₄ (sodium aluminate) Al(OH)₃ (aluminum hydroxide), Al₂O₃, psuedoboehmite, aluminum isopropoxide, Al(C₂H₃O₂)₃, AlBr₃, AlCl₃, Al(C₆H₅O₇) (aluminum citrate), AlF₃, Al(CHO₂)₃ (aluminum formate), Al₂(SO₄)₃, AlOOH (aluminum hydroxide oxide) and hydrates thereof.

9. The process according to claim 6, wherein the zinc source is selected from group consisting of elemental zinc Zn⁰, ZnSO₄, Zn(NO₃)₂, ZnBr₂, Zn(C₂H₃O₂)₂ (zinc acetate), ZnCO₃, Zn(CHO₂)₂ (zinc formate), ZnCl₂, zinc oxide, Zn⁰, ZnI₂ and hydrates thereof;
and
the optional fluoride source is selected from group consisting of HF, NH₄F, NaF, K₂SiF₆, KF and MgF₂ and
the optional hydroxide source is selected from the group consisting of potassium hydroxide, sodium hydroxide, lithium hydroxide, ammonium hydroxide, tripropylammonium hydroxide and tetramethyl ammonium hydroxide.

10. A pigment, comprising the synthetic zinc phlogopite platelet according to claim 1.

11. A pigment comprising the zinc phlogopite platelet according to claim 1,
(a) a layer of a dielectric material having a high index of refraction; and/or
(b) a metal layer.

12. The pigment according to claim 11 wherein the pigment further comprises in addition to layer (a) or (b) a metal oxide layer (c) of low refractive index and a layer (d) of high refractive index, wherein the difference of the refractive indices between the high and low refractive indexes is at least 0.1.

13. The pigment according to claim 11, wherein the metal oxide of layer (a) of high refractive index is TiO₂, ZrO₂, Fe2O3, Fe₃O₄, Cr₂O₃, ZnO, a mixture of these oxides, an iron titanate, an iron oxide hydrate, a titanium suboxide or a mixture and/or mixed phase of these compounds.

14. A paint, ink-jet, coatings, printing ink, plastic, cosmetic, glazes for ceramics and glass containing the pigments according to claim 10.
